(19) United States
(12) Patent Application Publication
(10) Pub. No.: US 2025/0221976 A1
(43) Pub. Date: Jul. 10, 2025

(54) COMPOUNDS, COMPOSITIONS AND METHODS OF TREATING NEUROPATHIC PAIN

(71) Applicants: THE UNIVERSITY OF HONG KONG, Hong Kong (CN); CITY UNIVERSITY OF HONG KONG, Hong Kong (CN)

(72) Inventors: Chi Wai CHEUNG, Hong Kong (CN); Zhiming SHAN, Hong Kong (CN); Jessica Aijia LIU, Hong Kong (CN)

(73) Assignees: THE UNIVERSITY OF HONG KONG, Hong Kong (CN); CITY UNIVERSITY OF HONG KONG, Hong Kong (CN)

(21) Appl. No.: 19/009,326

(22) Filed: Jan. 3, 2025

Related U.S. Application Data
(60) Provisional application No. 63/617,827, filed on Jan. 5, 2024.

(51) Int. Cl. A61K 31/4439; A61K 31/4245; A61P 25/00

(52) U.S. Cl. CPC A61K 31/4439; A61K 31/4245; A61P 25/00

(57) ABSTRACT

Disclosed is a novel compound 5-(3,4-difluorophenyl)-3-(6-methylpyridin-3-yl)-1,2,4-oxadiazole (DDO-7263, Compound 1), its derivatives, composition comprising the compound and method to effectively relieve nerve injury-induced neuropathic pain. Also disclosed are methods of treating diseases or disorders associated with the voltage-gated sodium channels. The disclosed methods may be utilized to treat diseases or disorders associated with pain in a subject, including nociceptive pain, chronic pain, neuropathic pain, and inflammatory pain. Also disclosed are inhibitors that specifically inhibit voltage-gated sodium channel function in a state-dependent manner. The disclosed compounds may also be used in pharmaceutical compositions and methods for treatment of pain associated with voltage-gated sodium channel activity.

### RELATED APPLICATIONS

This application claims priority to U.S. Provisional application Ser. No. 63/617,827 filed Jan. 5, 2024 which is incorporated by reference in its entirety.

### 1. FIELD

Disclosed are compounds, compositions and methods of treating pain, e.g., nerve injury-induced neuropathic pain.

### 2. BACKGROUND

Neuropathic pain caused by peripheral nerve lesion leads to long-term allodynia and hyperalgesia, presenting a significant challenge in pain management due to limited efficacy and undesirable side effects of available analgesics¹. Neuropathic pain is a debilitating chronic condition that poses a significant challenge of treatment. Current available analgesics are inadequate for pain relief with limited efficacy and undesirable side effects. Currently, antidepressants are used as one of primary medications for neuropathic pain, such as tricyclic antidepressants (TCAs) and serotonin-noradrenaline reuptake inhibitors, as well as anticonvulsants (e.g., pregabalin and gabapentin) that target calcium channels. However, these drugs lack of specificity and often affect multiple targets, resulting in numerous adverse effects, including cardiotoxicity, immune suppression, orthostatic hypotension, and nausea, which restrict their therapeutic usage. Thus, there is a vast unmet need for developing novel analgesics to target endogenous pain-resolution mechanisms and simultaneously modifying multiple pathophysiological mechanisms related to pain². The dorsal root ganglion (DRG), located on both sides of the spinal cord, act as a primary nervous system transmitting sensations from the peripheral nerve terminal to the central nervous system (CNS). Pathological changes in DRG upon nerve injury, such as hyperactive nociceptive neurons, are fundamental drivers for peripheral neuropathic pain (PNP) initiation and development³. The voltage-gated sodium (NaV) channels are integral to electrical signaling in DRG neurons, with nine isoforms of α-subunits (NaV1.1-1.9) identified in mammals⁴. These isoforms exhibit distinct expression patterns and variable channel properties. Genetic studies in human and animal have revealed that hyperactive NaV channels promote pain signaling 5.6. NaV1.7, encoded by the gene SCN9A in humans, is specifically expressed in the DRG and trigeminal ganglion⁷. As a large membrane ion channel, NaV1.7 produces a fast-activating, fast-inactivating, and slowly repriming current⁸, which functions as a threshold channel to generate and propagate action potentials by amplifying small sub-threshold depolarizations, a key mechanism for modulating neuronal excitability during nociceptive signaling transmission⁹. Multiple studies have linked the mutations in the NaV1.7 (SCN9A) gene to inherited pain conditions¹⁰, and eliminating NaV1.7 has demonstrated potent analgesic effects for PNP management¹¹. These findings suggested that targeting NaV1.7 represents a viable therapeutic strategy for treating PNP. Despite the development of NaV1.7 inhibitors was proceeded to clinical trials as a neuropathic pain therapy¹², the outcomes are unsatisfactory due to the disappointing pharmacokinetics and poor efficacy^{13, 14}. In light of these circumstances, preventing NaV 1.7 hyperactivation by suppressing its upstream effectors could be an alternative strategy for pain relief.

### 3. SUMMARY

The present disclosure relates to the fields of analgesic drug and natural compounds development for pain treatment.

DDO-7263 (herein designated “Compound 1”), a 1,2,4-oxadiazole derivative, exerts anti-inflammatory effects in neurodegenerative diseases, such as Parkinson's disease (PD) 15. It has now unexpectedly been discovered that 1,2,4-oxadiazole derivatives, e.g., Compound 1, have analgesic effects and are therefore useful in treating neuropathic pain. In certain embodiments, 1,2,4-oxadiazole derivatives, e.g., Compound 1, activate protective effector nuclear factor erythroid 2-related factor 2 (NRF2). In certain embodiments, 1,2,4-oxadiazole derivatives, e.g., Compound 1, inhibit voltage-gated sodium channels, in particular NaV1.7-mediated hyperactivation in the DRG.

The present disclosure provides the use of 1,2,4-oxadiazole derivatives, e.g., Compound 1 or a pharmaceutically-acceptable salt thereof in the preparation of a medicament for treating pain, e.g., neuropathic pain. The structural formula of Compound 1 is shown below:

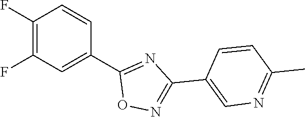 [SMILES: Cc1ccc(-c2noc(-c3ccc(F)c(F)c3)n2)cn1]

### Chemical Structure of DDO-7263 (Compound 1)

Disclosed is an application of 5-(3,4-difluorophenyl)-3-(6-methylpyridin-3-yl)-1,2,4-oxadiazole (DDO-7263, Compound 1) and related 1,2,4-oxadiazole derivatives in the preparation of a medicine for treating nerve lesion-induced neuropathic pain. Experimental results show that Compound 1 at a dosage of 50 μg/kg can relieve allodynia and hyperalgesia induced by a spared nerve injury (SNI) rodent model. Compound 1 can exert antinociceptive properties by activating the nuclear factor erythroid 2-related factor 2 (NRF2), a master regulator for antioxidative stress, and by selectively inhibiting voltage-gated sodium channel subtype 1.7 (NaV1.7), a voltage-gated sodium channel subtype predominantly expressed in sensory neurons, and specifically in the membrane of dorsal root ganglion neurons. These results demonstrate that Compound 1 and related 1,2,4-oxadiazole derivatives can have the effects of relieving peripheral neuropathic pain caused by nerve lesion.

Provided is a method for treating pain in a subject in need thereof, the method comprising administering to the subject an effective amount of Compound 1 or a pharmaceutically-acceptable salt thereof.

In certain embodiments, the pain is nociceptive pain.

In certain embodiments, the pain is inflammatory pain.

In certain embodiments, the pain is neuropathic pain. In certain embodiments, the neuropathic pain is peripheral neuropathic pain (PNP).

In certain embodiments, the pain is chronic pain.

In certain embodiments, the compound reduces or alleviates allodynia in the subject.

In certain embodiments, the compound reduces or alleviates hyperalgesia in the subject.

In certain embodiments, the pain is associated with a disease of condition such as, but not limited to, cancer, arthritis, osteoarthritis, diabetes, multiple sclerosis, autoimmune diseases (e.g., lupus, rheumatoid arthritis, Sjogren's syndrome, and Guillain-Barre syndrome), infections (e.g., shingles, Lyme disease, hepatitis B and C, leprosy, diphtheria, HIV, and polio), injuries (e.g., nerve trauma, spinal cord injury and stroke), metabolic diseases (e.g., kidney disease, liver disease, and hypothyroidism), postoperative pain, post-traumatic pain, post-thoracotomy pain, complex regional pain syndrome (CRPS), thalamic pain, paraplegic pain caused by myelopathy, chemotherapy induced polyneuropathy (CIPN), pain caused by radiation therapy, anesthesia dolorosa, phantom limb pain, small fiber neuropathy (SFN), hereditary motor and sensory neuropathies (HMSN), chronic inflammatory demyelinating polyneuropathy (CIDP), trigeminal neuralgia, post-herpetic neuralgia, intercostal neuralgia, entrapment neuropathies (e.g. carpal tunnel syndrome, tarsal tunnel syndrome, abdominal cutaneous nerve entrapment syndrome), sciatic pain, chronic idiopathic axonal polyneuropathy (CIAP), vulvodynia, proctodynia, lymphomatous neuropathy, myelomatous neuropathy, carcinomatous neuropathy, vasculitic/ischaemic neuropathy and other mono- and polyneuropathies.

In certain embodiments, the pain is associated with osteoarthritis.

In certain embodiments, the compound activates nuclear factor erythroid 2-related factor 2 (NRF2) in the subject.

In certain embodiments, the pain is associated with increased oxidative stress in the subject. In certain embodiments, the compound reduces or alleviates oxidative stress in the subject.

In certain embodiments, the pain is associated with an activated voltage-gated sodium channel. In certain embodiments, the compound inhibits activity of a voltage-gated sodium channel.

In certain embodiments, the compound binds to a voltage-gated sodium channel and has a dissociation constant (Kd) less than about 50 nM.

In certain embodiments, the compound inhibits the activity of a voltage-gated sodium channel with a half-maximal inhibitory concentration (IC50) of less than about 200 nM.

In certain embodiments, the voltage-gated sodium channel is NaV1.7.

In certain embodiments, the compound is administered orally, topically, intravenously or intrathecally.

In certain embodiments, the subject is administered a dose of the compound that is effective to achieve a concentration of the compound at the site of action of at least about 1 nM.

In certain embodiments, the subject is administered a daily oral dose of the compound of less than about 300 mg.

In certain embodiments, the subject is administered a dosage of about 50 μg/kg.

In other aspects provided is a method for treating pain in a subject in need thereof, the method comprising administering to the subject an effective amount of a 1,2,4-oxadiazole derivative that activates nuclear factor erythroid 2-related factor 2 (NRF2), reduces or alleviates oxidative stress in the subject, and/or or inhibits voltage-gated sodium channel (preferably NaV1.7) in the subject.

In certain embodiments, the 1,2,4-oxadiazole derivative is represented by the structure of formula (I):

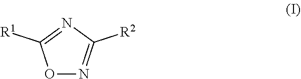

wherein
each of R¹ and R² is independently aryl, heterocyclyl or heteroaryl, each of which may be unsubstituted or substituted;
or a pharmaceutically-acceptable salt thereof.

In certain embodiments, the compound is represented by the structure of formula (1)

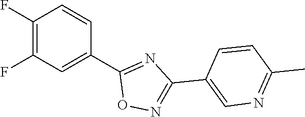 [SMILES: Cc1ccc(-c2noc(-c3ccc(F)c(F)c3)n2)cn1]

or a pharmaceutically-acceptable salt thereof.

In certain embodiments, the compound is selected from the group consisting of

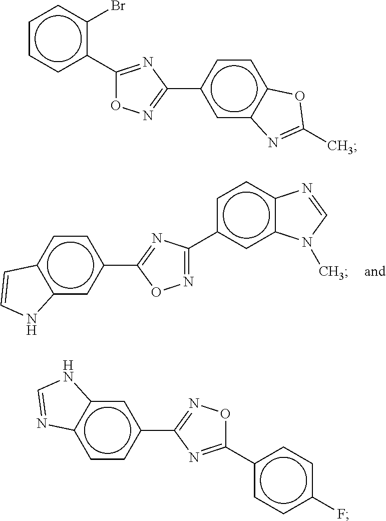

or a pharmaceutically-acceptable salt thereof.

In certain embodiments, the compound inhibits the activity of a voltage-gated sodium channel with half-maximal inhibitory concentration (IC50) of less than about 200 nM.

In certain embodiments, the voltage-gated sodium channel is NaV1.7.

### 4. BRIEF DESCRIPTION OF THE DRAWINGS

FIG. 1 is a graph of time course (A) and comparison of mechanical allodynia (B, von frey test), cold allodynia (C, acetone test), and hyperalgesia (D, pin test) of rats that underwent SNI surgery or sham control. At 7, 14, and 21 days after SNI injury, decreased mechanical allodynia, increased cold allodynia and hyperalgesia were noted. Representative immunoblot (E) and quantification (F) of NRF2 expression in L4-L6 DRG from naive, sham, and SNI rats. PWT, paw withdrawal threshold; BL, baseline; dpi, days post injury. Data are shown as mean±SEM; *p<0.05.

FIG. 2 is a graph of time course and comparison of mechanical allodynia (A, von frey test), cold allodynia (B, acetone test), and hyperalgesia (C, pin test) of rats that underwent SNI surgery. At 14 days after SNI injury, decreased mechanical allodynia, increased cold allodynia and hyperalgesia were noted. Rats were administered 50 μg/kg of Compound 1i.th. consecutively. PWT, paw withdrawal threshold; BL, baseline; dpi, days post injury. Data are shown as mean±SEM; *p<0.05.

FIG. 3 is a graph of representative immunoblot (A) and quantification (B) of NRF2 expression in L4-L6 DRG from SNI rats treated with vehicle or Compound 1 (DDO, 50 μg/kg), DRG tissues were collected 3 h after drug administration. Data are shown as mean±SEM; *p<0.05.

FIG. 4 is a graph of ROS levels in L4-L6 DRG from sham or SNI rats 3 h after vehicle or Compound 1 (DDO, 50 μg/kg, i.th) administration. (A) representative flow cytometry histogram for DCF staining. (B) normalized mean fluorescence intensity (MFI) for DCF staining. (C) representative flow cytometry gating analysis for DCF staining. Data are shown as mean±SEM; *p<0.05.

FIG. 5 is a graph of inhibition of Compound 1 on voltage-gated sodium (NaV) channels in DRG neurons. Representative NaV current traces (A), current-voltage curves (B), normalized peak currents (C), activation curve (D), and inactivation curve (E) from small diameter primary cultured DRG neurons, incubated with Compound 1 (DDO) 20 μM overnight. Data are shown as mean±SEM; *p<0.05.

FIG. 6 is a graph of inhibition of Compound 1 on NaV1.7 channels. Representative NaV1.7 traces (A), current-voltage curves (B), normalized peak NaV1.7 currents (picoamperes/picoFarads, pA/pF) (C), activation curve (D), and inactivation curve (E) recorded from HEK293 cells transfected with NaV1.7-EGFP plasmid, incubated overnight with or without Compound 1 (DDO) 20 μM. Data are shown as mean±SEM; *p<0.05.

FIG. 7 is a graph of inhibition of Compound 1 on NaV1.7 channels under excessive oxidative stress condition. Representative NaV1.7 current traces (A), summary of current-voltage curves (B), normalized peak NaV1.7 currents (C), activation curve (D), and inactivation curve (E) recorded from HEK293 cells transfected with NaV1.7-EGFP plasmid, incubated with H2O2 (500 μM, 20 min), followed by Compound 120 μM overnight treatment. Data are shown as mean±SEM; *p<0.05.

### 4.1 DEFINITIONS

As used herein the term “analgesic” may be defined as a drug designed to control pain. As used herein the term “chronic pain” may be defined as pain that persists for a period of time (e.g., greater than three months). In contrast to acute pain, chronic pain can continue after the injury or illness that caused it has healed or been resolved. Chronic pain may also occur in some subjects in the absence of apparent injury or illness.

As used herein the term “hyperexcitability” may be defined as a state of a neuron, or network of neurons, in which the likelihood that the neuron will be activated (depolarize) is increased. This may be due to the aberrant expression patterns of voltage-gated sodium channels after traumatic injury and inflammation. Hyperexcitability may lead to conditions such as chronic pain. Neurons that exhibit the state of hyperexcitability are said to be hyperexcitable.

As used herein the term “membrane depolarization” may be defined as a process of changing the electrical potential across a biological membrane. More typically, membrane depolarization refers to the process of neuronal depolarization wherein voltage-gated ion channels open on the surface of the neuronal membrane that rapidly change the electrical potential across the membrane of the neuron from about highly negative (−70 mV) to about zero (0 mV) or about slightly positive (+30 mV).

As used herein the term “nociceptive pain” may be defined as pain that is caused by the interpretation of signals sent by nociceptive receptors that sense potentially harmful stimuli.

As used herein the term “neuropathic pain” may be defined as pain caused by the damage to neurons or cells of the nervous system. Non-limiting examples of neuropathic pain include pain secondary to diabetic neuropathy or multiple sclerosis (MS).

As used herein the term “state-dependent accessibility” may be defined as a state in which voltage-gated sodium channels undergo changes in structural conformation when neuronal membranes are depolarized, transitioning from closed, to open, to inactivated, and back to closed during the course of membrane depolarization. Therefore, if the ability of a molecule to enter a specific site on a voltage-gated sodium channel depends on the state of the channel, i.e. closed, open, or inactivated, then the molecule displays state-dependent accessibility. This property is desirable in the context of design of analgesic drugs because it confers additional specificity to the drug action by, for example, only targeting voltage-gated sodium channels present on neurons that are in the process of depolarizing and are in the open conformation. This restricts drug action to aberrantly firing neurons, or neurons contributing to pain, and spares other neurons from inhibition.

As used herein the term “voltage-gated sodium channel” may be defined as integral membrane proteins that form ion channels and allow the passage of Na⁺ ions through a cell's plasma membrane in response to the increase of cellular membrane potential from about −70 mV to about −55 mV. voltage-gated sodium channel exist in three distinct conformational states: resting, open, and inactivated. Of these three conformational states, only open channels have the ability to conduct Na⁺ ions across the cell membrane.

A “subject in need thereof” as utilized herein may refer to a subject in need of treatment for a disease or disorder associated with hyperexcitability of sensory neurons and/or expression or activity of voltage-gated sodium channels. A subject in need thereof may include a subject having nociceptive pain, inflammatory pain, neuropathic pain, chronic pain, or pain associated with osteoarthritis. A subject in need thereof may also include a subject having chronic or chronic nociceptive pain.

The term “subject” may be used interchangeably with the terms “individual” and “patient” and includes human and non-human mammalian subjects.

The disclosed compounds, pharmaceutical compositions, and methods may be utilized to treat diseases and disorders associated with voltage-gated sodium channel activity and/or expression which may include, but are not limited to pain, chronic pain, chronic nociceptive pain. The disclosed compounds may be utilized to modulate the biological activity of voltage-gated sodium channels. The term “modulate” should be interpreted broadly to include “inhibiting” voltage-gated sodium channel biological activity including voltage-gated ion channel activity.

### 5. DETAILED DESCRIPTION

This invention provides the use of 1,2,4-oxadiazole derivatives, e.g., (5-(3,4-difluorophenyl)-3-(6-methylpyridin-3-yl)-1,2,4-oxadiazole (Compound 1) or a pharmaceutically-acceptable salt thereof for treating pain, e.g., neuropathic pain.

### 5.1 Compounds

In certain embodiments, the compound effective at treating pain is a 1,2,4-oxadiazole derivative or a pharmaceutically-acceptable salt thereof.

In certain embodiments, the 1,2,4-oxadiazole derivative activates nuclear factor erythroid 2-related factor 2 (NRF2). In certain embodiments, the 1,2,4-oxadiazole derivative reduces or alleviates oxidative stress in the subject. In certain embodiments, the 1,2,4-oxadiazole derivative inhibits voltage-gated sodium channel in the subject.

In some aspects, the compound is 5-(3,4-difluorophenyl)-3-(6-methylpyridin-3-yl)-1,2,4-oxadiazole (DDO-7263, Compound 1) or a pharmaceutically-acceptable salt thereof. The structural formula of Compound 1 is shown below:

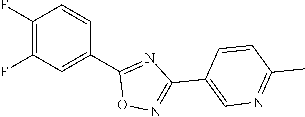 [SMILES: Cc1ccc(-c2noc(-c3ccc(F)c(F)c3)n2)cn1]

### Chemical Structure of DDO-7263 (Compound 1)

In other aspects, the 1,2,4-oxadiazole compound is represented by the structure of formula (I) or a pharmaceutically-acceptable salt thereof:

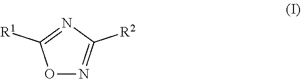

wherein
each of R¹ and R² is independently aryl, heterocyclyl or heteroaryl, each of which may be unsubstituted or substituted;
or a pharmaceutically-acceptable salt thereof

In yet other aspects, the 1,2,4-oxadiazole compound is represented by the structure of formula (I-a) or a pharmaceutically-acceptable salt thereof:

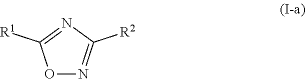

wherein
R¹ is an unsubstituted or substituted phenyl or heteroaryl; and
R² is an unsubstituted or substituted heteroaryl;
or a pharmaceutically-acceptable salt thereof.

The term “aryl” whether used alone or as part of another group, is defined herein as an aromatic group can be monocyclic or polycyclic. Non-limiting examples of aryl groups include phenyl, naphthyl, or biphenyl. The aryl group can be substituted or unsubstituted. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl. In certain embodiments, the aryl is phenyl. In certain embodiments, the aryl is 3,4-difluorophenyl.

The term “heterocycle” or “heterocyclyl”, whether used alone or as part of another group, is defined herein as any ring containing a ring atom that is not carbon, for example, N, O, S, P, Si, B, or any other heteroatom. A heterocycle can be aromatic (heteroaryl) or non-aromatic.

Non-limiting examples of heterocycles (heterocyclyl) include: diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolyl, pyrrolidinyl, morpholinyl, piperazinyl, piperidinyl, pyranyl, dihydropyranyl, tetrahydropyranyl, piperidin-2-onyl, 2,3,4,5-tetrahydro-1H-azepinyl, 1,2,3,4-tetrahydroquinoline hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from N, O, S, P, Si, B, and wherein further at least one of the rings that includes a heteroatom is aromatic. In heteroaryl groups that include 2 or more fused rings, the non-heteroatom bearing ring may be a carbocycle or aryl. One or more N or S atoms in a heteroaryl group can be oxidized to yield a compound such as, for example, N-pyridine oxide and thiole-1-oxide. In certain embodiments, each heteroaryl is independently a mono- or bi-cyclic group composed of from 5 to 10 ring members, and having at least one aromatic moiety and containing from 1 to 3 heteroatoms selected from oxygen, sulfur and nitrogen. In certain embodiments, each heteroaryl is independently (C3-C9) heteroaryl. In certain embodiments, each heteroaryl is a 5- or 6-membered heteroaryl. Non-limiting examples of heteroaryl include: pyridyl (including 2-pyridyl, 3-pyridyl, 4-pyridyl), 1H-indolyl, 2,3-dihydro-1H-indolyl, 1H-benzo[d]imidazolyl, benzo[d]oxazolyl, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, 4-dimethylaminopyridinyl; 7H-purinyl, 9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, and isoquinolinyl.

The aryl, heterocyclyl or heteroaryl described herein can be substituted or unsubstituted. Non-limiting examples of optional substituents include hydroxyl groups, sulfhydryl groups, halogens (halo), amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups. Other non-limiting examples of optional substituents include halogen, haloalkyl, hydroxy, alkoxy, haloalkoxy, cycloalkyl, aryl, heterocyclyl, heteroaryl, amido, alkylamido, dialkylamido, nitro, amino, cyano, azido, oxo, alkylamino, dialkylamino, carboxyl, thio, thioalkyl and thioaryl.

In certain embodiments, the substituent is a halogen. A halo group can be, for example, a chloro, bromo, fluoro, or iodo.

In certain embodiments, the substituent is an alkyl. Non-limiting examples of alkyl groups include straight and branched. Non-limiting examples of straight alkyl groups include methyl, ethyl, propyl, isopropyl, n-butyl, isobutyl, sec-butyl, tert-butyl, pentyl, hexyl, heptyl, octyl, nonyl, and decyl. Branched alkyl groups include any straight alkyl group substituted with any number of alkyl groups. Non-limiting examples of branched alkyl groups include isopropyl, isobutyl, sec-butyl, and t-butyl. Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl. In certain embodiments, the alkyl is a methyl.

In certain embodiments, In certain embodiments, R¹ in formula (I) or (I-a) is a phenyl. In certain embodiments, R¹ is a substituted phenyl. In certain embodiments, R¹ a 1H-indolyl. In certain embodiments, R¹ is a benzo[d]oxazolyl. In certain embodiments, R¹ is a 1H-benzo[d]imidazolyl. In certain embodiments, R¹ is phenyl substituted by one or more halogens. In certain embodiments, R¹ is phenyl substituted by one or more fluorine atoms. In certain embodiments, R¹ is phenyl substituted by one or more chlorine atoms. In certain embodiments, R¹ is phenyl substituted by one or more bromine atoms. In certain embodiments, R¹ is phenyl substituted by one or more iodine atoms. In certain embodiments, R¹ is 3,4-difluorophenyl.

In certain embodiments, R² in formula (I) or (I-a) is pyridyl, which can be 2-pyridyl, 3-pyridyl or 4-pyridyl. In certain embodiments, R² is a substituted pyridyl. In certain embodiments, R² is a pyridyl substituted with an alkyl. In certain embodiments, R² is a pyridyl substituted with methyl. In certain embodiments, R² is a 4-methyl-3-pyridyl. In certain embodiments, R² is a 1H-indolyl. In certain embodiments, R² is a 1H-benzo[d]imidazolyl. In certain embodiments, R² is a benzo[d]oxazolyl.

In some aspects, the compound is selected from the group consisting of

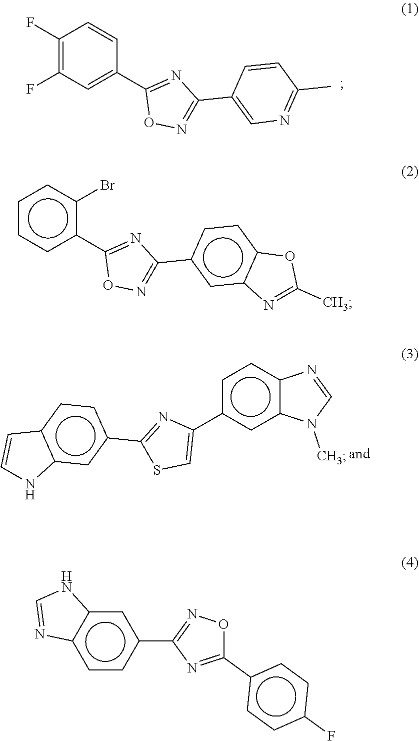

or a pharmaceutically-acceptable salt of any of the foregoing.

In certain embodiments, a compound described herein (e.g., Compound 1) is present as a free base. In certain embodiments, a compound described herein (e.g., Compound 1) is present as a pharmaceutically-acceptable salt.

As used herein, a “salt” is a salt of the present compound which has been modified by making acid or base, salts of the compounds. The salt may be pharmaceutically-acceptable.

The term “pharmaceutically acceptable salt” as used herein, refers to salts of the compounds, which are substantially non-toxic to living organisms, and include inorganic and organic acid or base addition salts of compounds of the present disclosure Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

These salts can be prepared in situ during the final isolation and purification of the compounds of the invention, or by separately treating a purified compound of the disclosure in its free base or free acid form with a suitable organic or inorganic acid or base, and isolating the salt thus formed.

Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts include, but are not limited to, are hydrobromide, hydrochloride, sulfate, bisulfate, phosphate, nitrate, valerate, oleate, palmitate, stearate, laurate, benzoate, lactate, phosphate, tosylate, citrate, sulfonate, maleate, fumarate, succinate, tartrate, naphthylate, mesylate, glucoheptonate, lactobionate, and laurylsulphonate, formate, tartrate, maleate, malate, citrate, tosylate, benzoate, salicylate, ascorbate, and the like. Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like.

Other examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like. The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

### 5.2 Pharmaceutical Compositions

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods disclosed herein may be administered as a single compound or a combination of compounds. For example, a compound that activates the biological activity of voltage-gated sodium channels may be administered as a single compound or in combination with another compound that activates the biological activity of voltage-gated sodium channels or that has a different pharmacological activity.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrating agents. Disintegrating agents are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### 5.3 Treatment of Pain

In certain aspects, the present disclosure provides a method for treating pain. In certain embodiments, the subject has nociceptive pain. In certain embodiments, the subject has inflammatory pain. In certain embodiments, the subject has neuropathic pain. In certain embodiments, the neuropathic pain is peripheral neuropathic pain (PNP). In certain embodiments, the subject has chronic pain. In certain embodiments, the subject has pain associated with an activated voltage-gated sodium channel.

In certain embodiments, the disclosed methods of treating pain can involve administering the compound orally. In other embodiments, the disclosed methods of treating pain may involve administering the disclosed compound topically. In certain embodiments, the disclosed methods of treating pain may involve administering the disclosed compound intravenously. In certain embodiments, the disclosed methods of treating pain may involve administering the disclosed compound intrathecally.

Pain can be chronic pain. Chronic pain may have no apparent cause or may be caused by a developing illness or imbalance. Chronic pain is defined as the disease of pain; its origin, duration, intensity and specific symptoms may vary. Moreover, chronic pain can be classified as either nociceptive or neuropathic.

Nociceptive pain includes tissue injury-induced pain and inflammatory pain such as that associated with arthritis, for example osteoarthritis.

Neuropathic pain, a debilitating chronic pain following nerve damage, is characterized by its chronic nature, hyperalgesia, or abnormal pain hypersensitivity to innocuous stimuli (tactile allodynia). Hyperalgesia is an exaggerated response to a painful stimulus. Allodynia is the perception of normal stimuli as painful (non-limiting examples include the touch of clothing, warm or cool air, etc.). Neuropathic pain can be a sequel to nerve damage in an extremity such as an arm, or a leg. Precipitating events can include trauma or amputations (e.g., phantom limb pain). Neuropathic pain can occur due to an adverse effect of drug therapies, or can occur as a component of disease pathologies, such as diabetes type 1 or type 2, shingles, HIV-1 infections, etc. Typically, neuropathic pain does not respond to opiates or non-steroidal anti-inflammatory drugs such as aspirin.

According to certain embodiments, the neuropathic comprises at least one symptom selected from hyperalgesia, allodynia, herpetic neuralgia, trigeminal neuralgia, trigeminal nerve pain, diabetic neuralgia, cancer pain, persistent postoperative or post-traumatic pain, post-thoracotomy pain, CRPS, pain associated with cancer, multiple sclerosis, AIDS, thalamic pain, paraplegic pain caused by myelopathy, anesthesia dolorosa and/or phantom limb pain.

Neuropathic (nerve) pain can be caused by damage, injury or dysfunction of nerves due to trauma, surgery, disease or chemotherapy. It is often described as burning, painful, cold or akin to electric shocks and may manifest with tingling, pins and needles, numbness or itching. Neuropathic pain can be the primary symptom of a particular condition or disease state, such as cancer, complex regional pain syndrome or post herpetic neuralgia. It can also be associated with other medical conditions or other forms of pain, including pelvic pain, fibromyalgia and orofacial pain. Phantom pain following a limb amputation is also a type of neuropathic pain.

As used herein, the term “allodynia” refers to a pain experienced with a normally painless stimuli, such as cold, pressure, or brushing against the skin. There are several types of allodynia, including mechanical allodynia (also known as tactile allodynia), thermal allodynia (also known as hot or cold allodynia), and movement allodynia. Mechanical allodynia is characterized by pain in response to a mechanical interaction (e.g., static mechanical allodynia: pain in response to touch; or dynamic mechanical allodynia-pain in response to stroking). Thermal allodynia is characterized by pain experienced from normally mild skin temperatures in the affected area. Movement allodynia is characterized by pain triggered by normal movement of joint or muscles.

As used herein, the term “hyperalgesia” means an abnormally increased sensitivity to pain. Hyperalgesia is characterized by extreme or increased pain sensation caused by a normally painful stimuli, e.g., heat or pinpricks. Hyperalgesia can be primary (i.e., pain sensitivity that occurs directly from damaged tissues) or secondary (i.e., pain sensitivity that occurs in surrounding undamaged tissues). In certain embodiments, primary or secondary hyperalgesia can develop from long-term use of opioids.

In certain embodiments, neuropathic pain encompasses “peripheral neuropathic pain” as well as “central neuropathic pain” which is generally defined as pain arising as a direct or indirect consequence of a lesion or disease affecting the peripheral somatosensory system. Peripheral neuropathic pain includes all types of peripheral neuropathic pain, caused by for instance peripheral diabetic neuropathy type 1 or 2, induced by various noxious substances such as alcohol, caused by various deficiencies such as vitamin B1, B6 and/or B12 deficiency, various intoxications, such as hypervitaminosis B6, caused by hypothyroidism, or chemotherapy induced polyneuropathy (CIPN). CIPN can be caused from chemotherapeutic agents such as: alkylating agents (including but not limited to cis-platinum (II)-diaminedichloride (platinol or cisplatin), oxaliplatin, and carboplatin); antitumour antibiotics (including but not limited to anthracyclines such as doxorubicin); antimetabolites (including but not limited to folic acid analogues such as pyrimidine analogues such as 5-fluorouracil (Fluoruracil, 5-FU), gemcitabine); histone deacetylase inhibitors (HDI) for instance, Vorinostat; natural alkaloids, including paclitaxel; inhibitors of protein tyrosine kinases and/or serine/threonine kinases including Sorafenib, Erlotinib, or Dasatanib.

Other peripheral neuropathies that can cause peripheral neuropathic pain include: small fiber neuropathy (SFN), hereditary motor and sensory neuropathies (HMSN), chronic inflammatory demyelinating polyneuropathy (CIDP), trigeminal neuralgia, post-herpetic neuralgia, intercostal neuralgia, entrapment neuropathies (e.g. carpal tunnel syndrome, tarsal tunnel syndrome, abdominal cutaneous nerve entrapment syndrome), sciatic pain, chronic idiopathic axonal polyneuropathy (CIAP), vulvodynia, proctodynia, neuropathy due to infectious disease conditions, such as post-polio syndrome, AIDS or HIV-associated, lyme associated, Sjogren syndrome-associated, lymphomatous neuropathy, myelomatous neuropathy, carcinomatous neuropathy, vasculitic/ischaemic neuropathy and other mono- and polyneuropathies.

In some aspects, provided is a method for treating pain in a subject in need thereof, the method comprising administering to the subject an effective amount of a 1,2,4-oxadiazole derivative that activates nuclear factor erythroid 2-related factor 2 (NRF2) in the subject.

In other aspects, provided is a method for treating pain in a subject in need thereof, the method comprising administering to the subject an effective amount of a 1,2,4-oxadiazole derivative that reduces or alleviates oxidative stress in the subject.

In other aspects, provided is a method for treating pain in a subject in need thereof, the method comprising administering to the subject an effective amount of a 1,2,4-oxadiazole derivative that inhibits voltage-gated sodium channel is NaV1.7 in the subject.

The pharmaceutical compositions may be utilized in methods of treating a disease or disorder associated with the biological activity of voltage-gated sodium channels. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with biological activity of voltage-gated sodium channels. The disclosed methods may include administering an effective amount of the disclosed compounds for treating a nociceptive pain, inflammatory pain, neuropathic pain, chronic pain, or pain associated with osteoarthritis. The disclosed methods may also include administering an effective amount of the disclosed compounds for treating chronic or chronic nociceptive pain.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

Oral administration is an illustrative route of administering the compounds employed in the compositions and methods disclosed herein. Other illustrative routes of administration include transdermal, percutaneous, intravenous, intramuscular, intranasal, sublingual, buccal, intrathecal, intracerebral, vaginal, rectal, ocular, respiratory (via inhalation), or intrarectal routes, . . . . The route of administration may be varied in any way, limited by the physical properties of the compounds being employed and the convenience of the subject and the caregiver.

In certain embodiments, the compounds are administered directly into the CNS, for example by intralumbar injection or interventricular infusion of the compounds directly into the cerebrospinal-fluid (CSF), or by intraventricular, intrathecal or interstitial administration.

### 5.4 Dosing and Dosing Schedules

In accordance with the methods of the present disclosure, any of these compounds can be administered to the subject in an amount effective to activate nuclear factor erythroid 2-related factor 2 (NRF2) in the subject, reduce or alleviates oxidative stress in the subject inhibits voltage-gated sodium channel is NaV1.7 in the subject

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

In other embodiments, a suitable amount of the compounds ranges from about 1 to about 500 mg per day, for example about 10 mg per day, about 20 mg per day, about 30 mg per day, about 40 mg per day, about 50 mg per day, about 60 mg per day, about 70 mg per day, about 80 mg per day, about 90 mg per day, about 100 mg per day, about 120 mg per day, about 140 mg per day, about 160 mg per day, about 180 mg per day, about 200 mg per day, about 220 mg per day, about 240 mg per day, about 260 mg per day, about 280 mg per day, about 300 mg per day, about 320 mg per day, about 340 mg per day, about 360 mg per day, about 380 mg per day, about 400 mg per day, about 420 mg per day, about 440 mg per day, about 460 mg per day, about 480 mg per day, about 500 mg per day or higher or lower.

A compound described herein can be present in a composition in a range of from about 1 mg to about 500 mg; from about 1 mg to about 200 mg; from about 1 mg to about 100 mg; from about 5 mg to about 500 mg, from about 5 mg to about 500 mg, from about 5 mg to about 100 mg, from about 10 mg to about 50 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg or higher, from about 350 mg to about 400 mg, from about 400 mg to about 450 mg, from about 450 mg to about 500 mg, or higher or lower.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 100 mg, about 125 mg, about 150 mg, about 175 mg, about 200 mg, about 250 mg, about 300 mg, about 350 mg, about 400 mg, about 450 mg, or about 500 mg, or higher or lower.

In certain embodiments, a dose can be expressed in terms of an amount of the drug divided by the mass of the subject, for example, milligrams of drug per kilograms of subject body mass. In certain embodiments, a compound is administered in an amount ranging from about 0.001 mg/kg to about 1,000 mg/kg, about 0.001 mg/kg to about 1,000 mg/kg, about 0.001 mg/kg to about 100 mg/kg, about 0.001 mg/kg to about 10 mg/kg, about 0.001 mg/kg to about 5 mg/kg, about 0.001 mg/kg to about 1 mg/kg, about 0.001 mg/kg to about 0.5 mg/kg, about 0.001 mg/kg to about 0.1 mg/kg, about 0.001 mg/kg to about 0.05 mg/kg, about 1 mg/kg to about 1,000 mg/kg, about 1 mg/kg to about 500 mg/kg, about 1 mg/kg to about 250 mg/kg, about 1 mg/kg to about 100 mg/kg, or about 1 mg/kg to about 50 mg/kg. In certain embodiments, a compound is administered in an amount of about 1 μg/kg, about 2 μg/kg, about 5 μg/kg, about 10 μg/kg, about 20 μg/kg, about 50 μg/kg, about 100 μg/kg, about 150 μg/kg, about 200 μg/kg, about 250 μg/kg, about 300 μg/kg, about 350 μg/kg, about 400 μg/kg, about 450 μg/kg, about 500 μg/kg, about 550 μg/kg, about 600 μg/kg, about 650 μg/kg, about 700 μg/kg, about 750 μg/kg, about 800 μg/kg, about 850 μg/kg, about 900 μg/kg, about 950 μg/kg or about 1,000 μg/kg, about 2 mg/kg, about 5 mg/kg, about 10 mg/kg, about 20 mg/kg, about 50 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, of a subject's body mass.

In certain embodiments, a dose can be expressed in terms of an amount of the drug divided by the mass of the subject per day, for example, milligrams of drug per kilograms of subject body mass, per day (mg/kg/day/day). In certain embodiments, a compound is administered in an amount ranging from about 0.001 mg/kg/day to about 1,000 mg/kg/day, about 0.001 mg/kg/day to about 1,000 mg/kg/day, about 0.001 mg/kg/day to about 100 mg/kg/day, about 0.001 mg/kg/day to about 10 mg/kg/day, about 0.001 mg/kg/day to about 5 mg/kg/day, about 0.001 mg/kg/day to about 1 mg/kg/day, about 0.001 mg/kg/day to about 0.5 mg/kg/day, about 0.001 mg/kg/day to about 0.1 mg/kg/day, about 0.001 mg/kg/day to about 0.05 mg/kg/day, about 1 mg/kg/day to about 1,000 mg/kg/day, about 1 mg/kg/day to about 500 mg/kg/day, about 1 mg/kg/day to about 250 mg/kg/day, about 1 mg/kg/day to about 100 mg/kg/day, or about 1 mg/kg/day to about 50 mg/kg/day. In certain embodiments, a compound is administered in an amount of about 1 μg/kg/day, about 2 μg/kg/day, about 5 μg/kg/day, about 10 μg/kg/day, about 20 μg/kg/day, about 50 μg/kg/day, about 100 μg/kg/day, about 150 μg/kg/day, about 200 μg/kg/day, about 250 μg/kg/day, about 300 μg/kg/day, about 350 μg/kg/day, about 400 μg/kg/day, about 450 μg/kg/day, about 500 μg/kg/day, about 550 μg/kg/day, about 600 μg/kg/day, about 650 μg/kg/day, about 700 μg/kg/day, about 750 μg/kg/day, about 800 μg/kg/day, about 850 μg/kg/day, about 900 μg/kg/day, about 950 μg/kg/day or about 1,000 μg/kg/day, about 2 mg/kg/day, about 5 mg/kg/day, about 10 mg/kg/day, about 20 mg/kg/day, about 50 mg/kg/day, about 100 mg/kg/day, about 150 mg/kg/day, about 200 mg/kg/day, about 250 mg/kg/day, about 300 mg/kg/day, about 350 mg/kg/day, about 400 mg/kg/day, about 450 mg/kg/day, about 500 mg/kg/day, of a subject's body mass.

In certain embodiments, the subject is administered a dose of the compound that is effective to achieve a concentration of the compound at the site of action of at least about 1 nM to about 100 μM. For example, the subject is administered a dose of the compound that is effective for achieving a concentration of the compound at the site of action of about 20 μM.

In certain embodiments, the subject is administered a daily intrathecal injection dose of the compound of 50 μg/kg, or within a range bounded by any of these values.

In certain embodiments, the compound inhibits the activity of a voltage-gated sodium channel and has about a 50% inhibitory rate of about 20 μM.

In certain embodiments, the binds to and/or inhibits an activated voltage-gated sodium channel. In one embodiment, the activated voltage-gated sodium channel is NaV1.7.

### 6. EXAMPLES

### 6.1 Materials and Methods

### 6.1.1 Animals and Ethics Approvals

All experiments involving animals were reviewed and approved by local Institutional animal ethics committees (Committee on the Use of Live Animals in Teaching and Research (CULATR) of the University of Hong Kong and conducted in accordance with relevant national and international regulations (International Association for the Study of Pain Guidelines for the Use of Animals in Research; The Cap. 340 Animals (Control of Experiments) Ordinance and Regulations in Hong Kong). Adult Sprague-Dawley rats (age 6-8 weeks on arrival) for in vivo experiments or tissue collection were sourced either from Beijing Viton Lever Co. Ltd or local breeding colonies. Animals were housed in groups of two or three per cage under a temperature (23±3° C.) and light (12 hours light/dark cycle)-controlled room.

### 6.1.2 Spared Nerve Injury (SNI) Surgery

The SNI surgery was constructed as previously described^{16, 17}. In brief, after animals were anesthetized by isoflurane (1.5-2.5%), a half centimeter incision was made in the middle of the outer. Muscles from the upper layer were bluntly dissected to expose the sciatic nerve and its branches to the left hind leg's common peroneal, tibial, and peroneal nerves. Then, the front two nerves were clipped, and the peroneal nerve was preserved. In the sham-operated group, the sciatic nerve was only exposed, and no clipping or injury was done. After suturing the skin, animals were transferred to an observation box for recovery.

### 6.1.3 Intrathecal Injection

Intrathecal administration of vehicle or drugs was conducted by direct lumbar puncture in the L5-L6 intervertebral space with a 30-gauge needle following the method of Mestre¹⁸. This procedure was performed under isoflurane anesthesia to permit rapid recovery of the animals. The quality of each injection was ensured by the observation of an injection-induced tail-flick.

### 6.1.4 Behavioral Assessment

### Mechanical Allodynia

Assessment of mechanical allodynia was using the Von Frey test as previously described¹⁷. The animals were placed in plastic cages with metal mesh on the bottom and allowed to acclimatize for at least half an hour before each test. Each Von Frey wire was held perpendicular to the contact surface of the paw until it bent. Tests were carried out and data collected using Dixon's “up and down” method (successive increases and decreases in stimulus intensity)¹⁹

### Cold Allodynia

Assessment of cold allodynia was using acetone test²⁰. Animals were placed in the assessment cage described above for habituation. Acetone was collected by pipette for each trial. Every drop of acetone (a volume of 200 μL for rat, and 20 μL for mice) was then gently delivered to the plantar of the ipsilateral hind paw, avoiding needle contact with the skin. As the acetone fast spread across the plantar skin and evaporated, noxious withdrawal responses of the hind paw (fluttering, licking, or hanging) against the cold stimuli was recorded. The trial was repeated 5 times for each animal, and data was presented as percentage of noxious withdrawal response out of 5 trials.

### Hyperalgesia

Hyperalgesia was evaluated by pin test described previously²⁰. Briefly, after animals were adapted to the environment of assessment cage, a 22G anesthesia spinal needle (BD, Franklin Lakes, NJ) was applied to indent to the plantar skin of the ipsilateral hind paw vertically and gradually without rapid puncture. Normal response (brief flinch) or noxious-like response (sustained elevation of the paw with licking, shaking, or grooming) was recorded in 10 repeated trials with a minimum of 10 seconds' gap. Data was expressed as percentage of noxious-like response out of 10 trials.

### 6.1.5 Dorsal Root Ganglion (DRG) Neuron Isolation and Culture

DRG neurons were obtained from rodents by acute isolation as previously described^{21, 22}. After animals were euthanized, the dorsal skin was removed to perform laminectomy to expose DRG, L4-L6 DRGs were rapidly collected from the intervertebral foramen. The collected DRGs were digested in 3 mL bicarbonate-free, serum-free, sterile DMEM (cat. no. 11965; Thermo Fisher Scientific) containing a mixture of enzymes (neutral protease, 3.125 mg/mL (cat. no. LS02104; Worthington) and collagenase Type I 5 mg/mL (cat. no. LS004194; Worthington)), incubated in a shaker at 37° C. After 40 minutes incubation, dissociated DRG neurons were then gently centrifuged (800 r/min, 3 min) to collect cells, washed and resuspended with DRG medium (DMEM containing 10% fetal bovine serum and 1% penicillin/streptomycin). Subsequently, DRG neurons were inoculated on glass crawl sheets previously treated with poly-d-lysine (50 μg/mL). DRG neurons were cultured in the mixed medium and subjected to the following experiments.

### 6.1.6 Cell Lines and Culture

HEK293 cells were purchased from Shanghai Institute of Biological Sciences, Chinese Academy of Sciences. HEK293 cells were cultured in medium (DMEM containing 10% fetal bovine serum and 1% penicillin/streptomycin) in an atmosphere of 5% CO2 at 37° C.

### 6.1.7 Plasmids and Transfection of HEK293 Cell Line

The NaV1.7 sequence was synthesized by GenScript (Nanjing, Jiangsu Province, China) and inserted into the pcDNA3.1 (+)-C-DYK plasmid between the BamH1 and EcoRI restriction sites. The empty plasmid of pcDNA3.1 purchased from Shenzhen Yanming Biotechnology Company was used as a mock transfection control.

For transfection, HEK293 cells were seeded at 60-70% confluency in 9 mm cell slide and transfected with JetPRIME (Polyplus-transfection SA, France) according to the manufacturer's protocols. HEK293 cells were transfected for 6-8 hours using pcDNA3.1-NaV1.7-Flag plasmid. Subsequently, the medium was replaced, compounds were added and incubated overnight for whole-cell patch recording.

### 6.1.8 Whole-Cell Voltage Clamp Electrophysiology

Whole-cell voltage clamp recording was performed as previously described. Briefly, cell patch was conducted under a HEKA EPC 10-USB patch clamp amplifier (HEKA Instruments). Data were obtained by Patchmaster (HEKA) and analyzed with Fitmaster (HEKA). All experiments were performed at room temperature. The composition of the extracellular recording solution used to record NaV currents was (mM): 140 NaCl, 30 tetraethylammonium chloride, 10 D-glucose, 3 KCl, 1 CaCl₂), 0.5 CdCl₂, 1 MgCl₂, and 10 HEPES, and the pH of the solution was adjusted with NaOH to 7.3 with an osmolarity of 310-315 mOsm/L. The composition of the intracellular recording solution was (mM): 140 CsF, 10 NaCl, 15 HEPES and 1.1 Cs-EGTA, and the pH of the solution was adjusted to 7.3 using CsOH.

NaV currents were measured from rodent DRG small diameter (<30 μm) neurons. The voltage protocols were as follows: (i) I-V protocol: DRG neurons were clamped at −80 mV, and the cells were depolarized in 200 ms steps in the range of −70 to +60 mV (+5 mV increments). This was done to obtain a current density such that the activation of NaV channel was generated between approximately 0-10 mV. This process occurs between 0 and 10 mV and can be analyzed as a function of voltage, as inferred from the peak current density (normalized to the cell capacitance (picofarad, pF)); (ii) Inactivation protocol: starting from a clamp potential of −60 mV, a hyperpolarization/depolarization pulse of 1 second in the range of −120 to 20 mV, in steps of +10 mV for 1 second. This incremental increase in membrane potential puts a different proportion of NaV channel into a state of rapid deactivation—in this case, a test pulse of 0-mV for 200 ms shows rapid deactivation when normalized to the maximum NaV current. (iii) Statistic of biophysical properties of activation and inactivation: after converting the current values to conductance (G), the conductance-voltage relationship was fitted with a Boltzmann equation. The G value for each neuron was normalized to the maximal value (Gmax) derived from the fit. The Boltzmann relevant factors, similar to half-maximal activation (V50) and slope, for the single fits to the data, are calculated, summarized, and compared among groups. Inhibition of NaV1.7-mediated currents was assessed from HEK293 cell lines expressing the NaV1.7 subunits.

### 6.1.9 Immunoblotting

For western blotting, L4-6 DRGs were extracted from the ipsilateral surgery of SNI rats and stored in liquid nitrogen to be set aside. DRGs were then sheared in pre-cooled potent RIPA lysate (containing protease and phosphatase inhibitors) and their whole cell lysates were obtained by ultrasonic crushing on ice. Whole-cell protein extracts were separated using 8% SDS-PAGE gels and transferred to PVDF membranes (activated with methanol). Protein blots were closed in 5% skimmed milk (Tris buffer configuration containing 0.1% Tween-20) for 1 h, then incubated overnight with target primary antibodies or β-Actin antibody (Cell Signaling, 5174, 1:1000), respectively, and the next day for 1 h with the corresponding secondary antibody. Immunoreactive bands were detected by chemiluminescence.

### 6.1.10 Reactive Oxygen Species (ROS) Level Measurement

ROS level of DRG neurons were labeled by ROS Assay Kit (Beyotime Co., Ltd., China) and detected by flow cytometry according to the manufacturer's instructions. Briefly, 2′,7′-dichlorodihydrofluorescein diacetate (DCFH-DA) probe was diluted 1:1000 in serum-free culture medium to a final concentration of 10 μM. Acutely isolated DRG neurons were resuspend in diluted DCFH-DA to a final cell density of 5×106 cells/ml. Incubate in 37° C. for 20 minutes. The cell resuspension was mixed every 3-5 minutes to ensure the adequate contact between probes and cells, and then the cells were washed three times with serum-free culture medium to remove the residual DCFH-DA thoroughly. DCFH-DA itself is cell-permeable but non-fluorescent. However, after entering the cell, DCFH-DA is hydrolyzed by cellular esterase to generate DCFH (2′,7′-dichlorodihydrofluorescein), which is non-permeable to the cell membrane, and can be oxidized by ROS into fluorescent DCF (2′,7′-dichlorofluorescein) that can be detected by flow cytometry. The fluorescence intensity represents the intracellular ROS levels.

### 6.1.11 Data Analysis

Unless stated otherwise, data are presented as mean±S.E.M. throughout. All measurements were taken from distinct samples. Data were plotted and analyzed using GraphPad Prism version 9.4.1, as detailed throughout. Statistical significance was defined as p<0.05.

### 6.2 Results

### (1) Insufficient NRF2 Activation in DRG Contributes to Neuropathic Pain

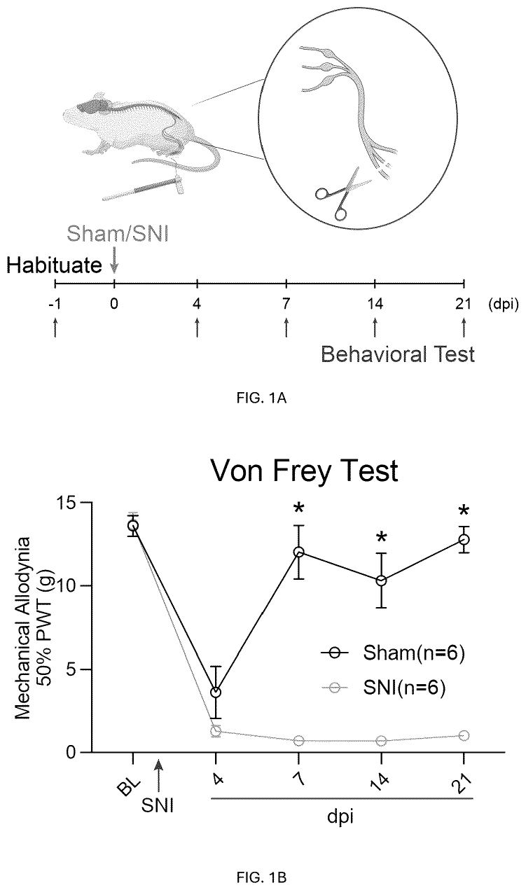

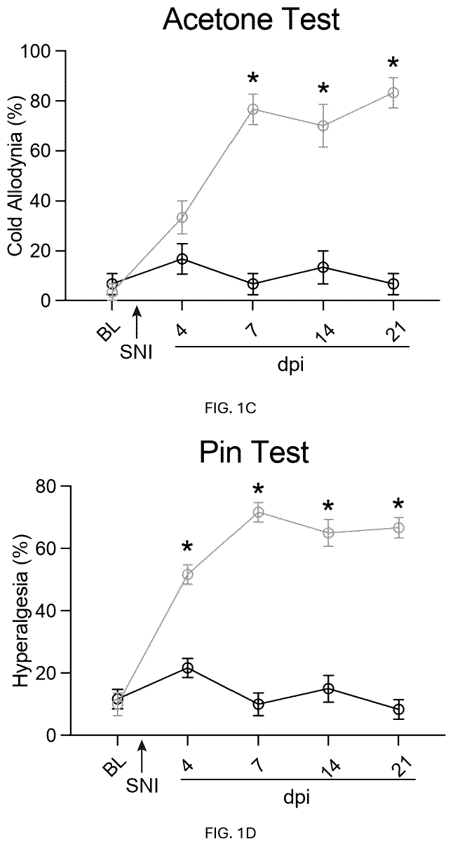

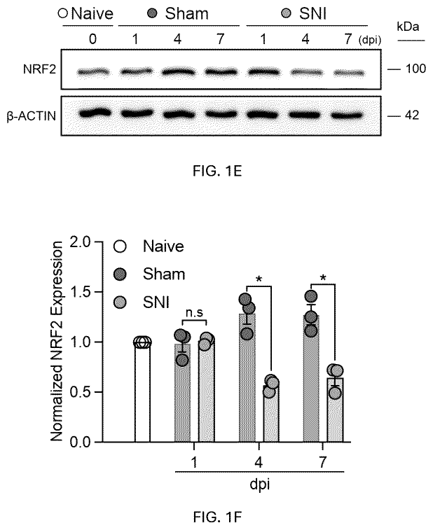

To establish the link between oxidative stress in DRG neurons and neuropathic pain development, a spared nerve injury (SNI) model was used. The SNI model mimics the most common clinical presentation of neuropathic pain in human patients, in which the peroneal and tibial nerves are injured to induce long-term neuropathic pain (FIG. 1A). Consistently, the SNI rats developed sustained mechanical allodynia, cold allodynia, and hyperalgesia since the 7th day post injury (dpi) (FIG. 1B-D). Coinciding with the occurrence of acutely evoked nocifensive behavior, such as transient mechanical allodynia, a gradual increase in NRF2 expression was observed in the ipsilateral DRG (L4-L6) of the sham group compared to naive animals (FIG. 1E, F). However, instead of overexpression, NRF2 was significantly suppressed in the DRG of SNI rats since the 4th day after the nerve injury (FIG. 1E, F). These findings suggest a possible algesic effect of insufficient NRF2 in the DRG that contributes to neuropathic pain.

### (2) Compound 1 Alleviates Mechanical Allodynia, Cold Allodynia, and Hyperalgesia in Rodent Model of Neuropathic Pain.

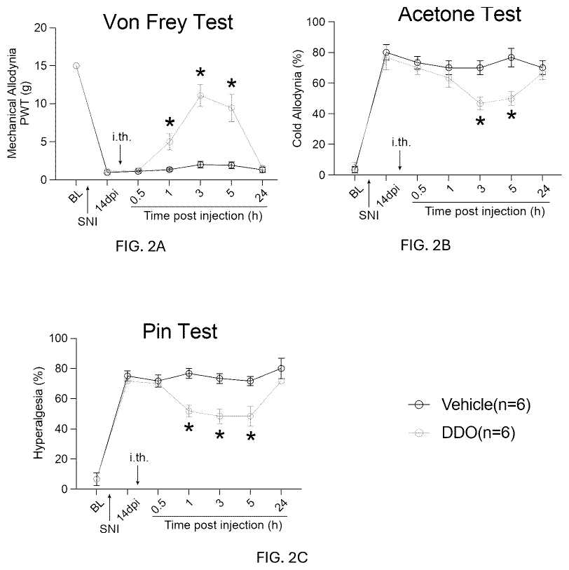

As a novel NRF2 activator, Compound 1 presented potent NRF2 activating and antioxidative properties in nervous system¹⁵. However, the analgesic properties of Compound 1 have not been reported. The present disclosure utilized a spared nerve injury (SNI) rat model that mimics the most common clinical presentation of neuropathic pain in human patients, in which the peroneal and tibial nerves are injured to induce long-term neuropathic pain. Consistently, the SNI rats developed sustained mechanical allodynia, cold allodynia, and hyperalgesia since the 14th day post injury. These rats were then treated with Compound 1 50 μg/kg/day intrathecally (i.th), and evaluated their pain behavioral in three different types of tests: Von Frey test for mechanical allodynia, acetone test for cold allodynia, and pin test for hyperalgesia. The results demonstrate that treatment with Compound 1 resulted in potent antinociceptive abilities against neuropathic pain in SNI rats (about 70.0% max inhibiting rate in mechanical allodynia, 35.0% in cold allodynia, and 37.5% in hyperalgesia, FIG. 2). These results indicated that Compound 1 is a potential analgesic in neuropathic pain therapy.

### (3) Compound 1 Increases Expression Level of NRF2 in DRG Cells.

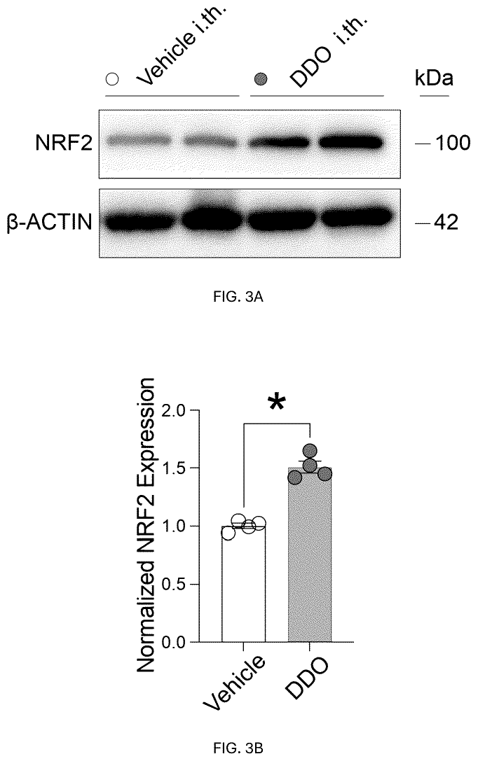

The present disclosure demonstrates that Compound lexerts its antinociceptive properties through NRF2 activation. The expression level of NRF2 was evaluated in in rats DRG after Compound 1 administration. Compound 1 increased the expression level of NRF2 in SNI rat's L4-L6 DRGs (FIG. 3). These findings indicate that Compound 1 may exerts NRF2 antinociceptive properties through NRF2 activation.

### (4) Compound 1 Improves Excessive Oxidative Stress in DRG Cells.

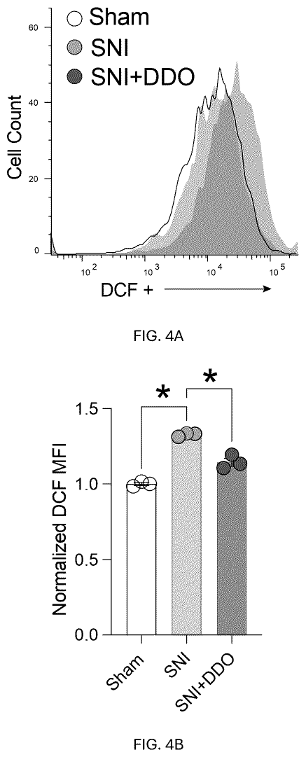

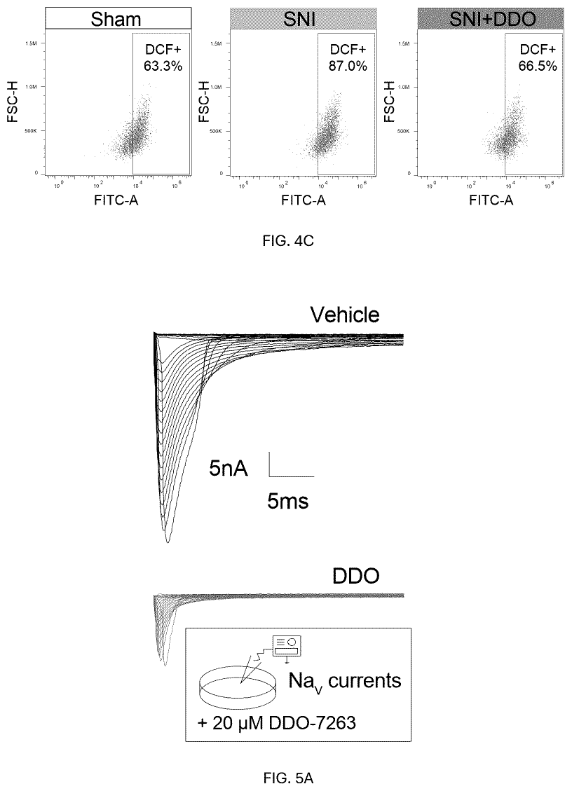

As a master regulator for redox homeostasis, NRF2 is a transcriptional factor that modulates oxidative stress level in tissues. It was investigated whether Compound 1 helps to rebalance the excessive oxidative stress condition in DRG cells after SNI. To detect the oxidative stress levels in the DRG, the 2′,7′-dichlorodihydrofluorescein diacetate (DCFH-DA) ROS detector was used to quantify the amount of 2′,7′-dichlorofluorescein (DCF) positive cells in rats treated with or without Compound 1. DCFH-DA can be hydrolyzed by the intracellular esterase to generate 2′,7′-dichlorodihydrofluorescein (DCFH), which can be further oxidized by ROS to generate fluorescent DCF. Based on flow cytometry analysis, the SNI group exhibited a higher ROS level in DRG neurons than the Sham group, while such increased ROS level was decreased after Compound 1 treatment (FIG. 4). These results show that treatment with Compound 1 improved the excessive oxidative stress condition in DRG cells.

### (5) Compound 1 Selectively Inhibits NaV1.7 Current Density in DRG Neurons.

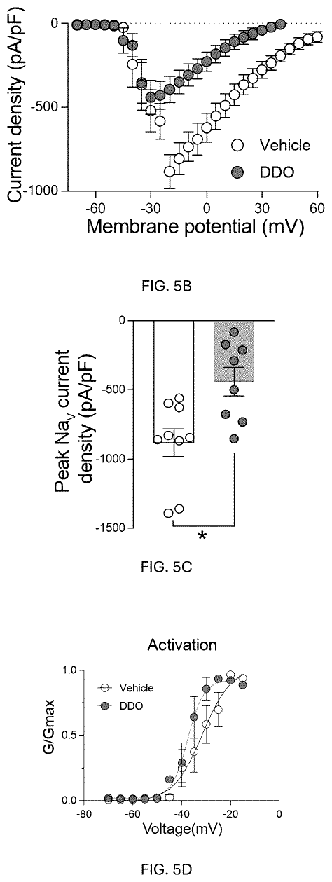

As important pain targets, voltage-gated sodium (NaV) channels are pore-forming transmembrane proteins that are critical for initiating and propagating action potentials in nociceptive neurons. Accumulating studies support the functional links among oxidative stress, ROS, and NaV channels^{23, 24, 25}. It was investigated whether Compound 1 alleviates neuropathic pain through modulating NaV channels' function. Whole-cell patch clamp recording was performed on primary cultural small diameter (<30 μm) nociceptive DRG neurons to identify functional changes of NaV channels during Compound 1 administration. A significant inhibition of NaV currents in Compound 1 treated group was observed (FIG. 5).

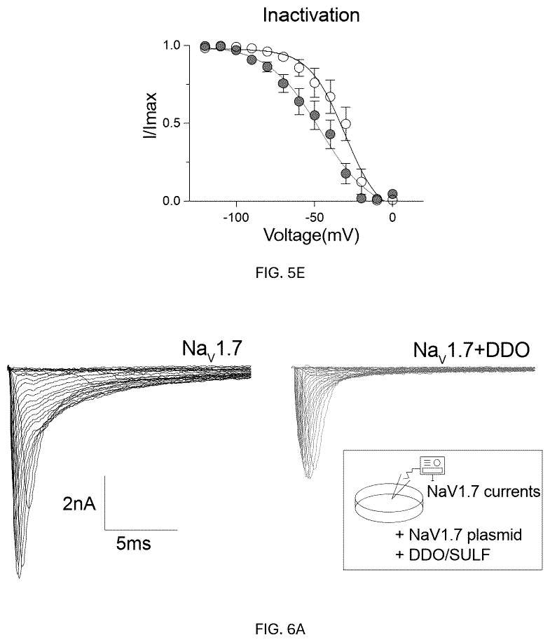

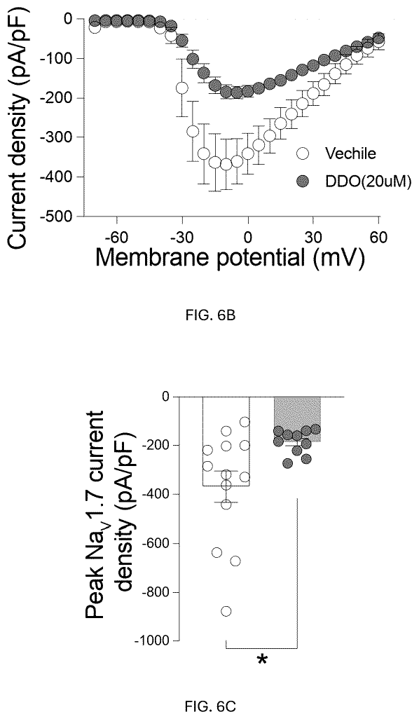

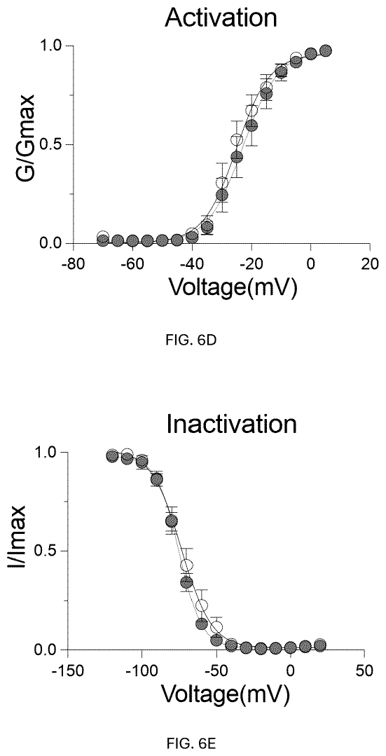

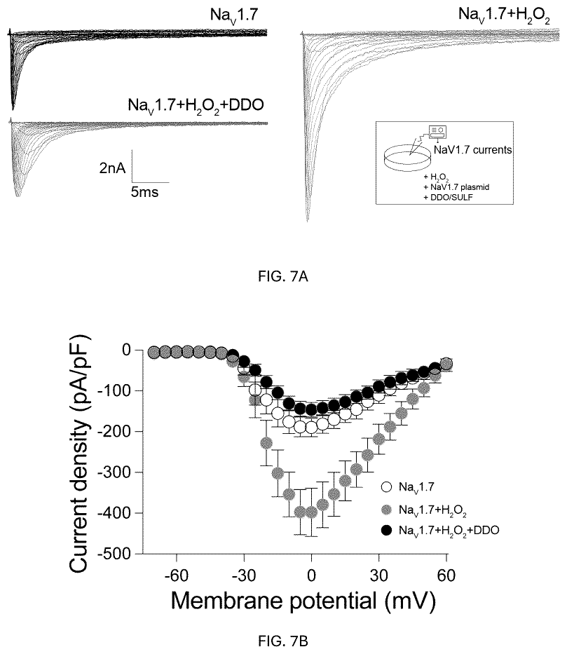

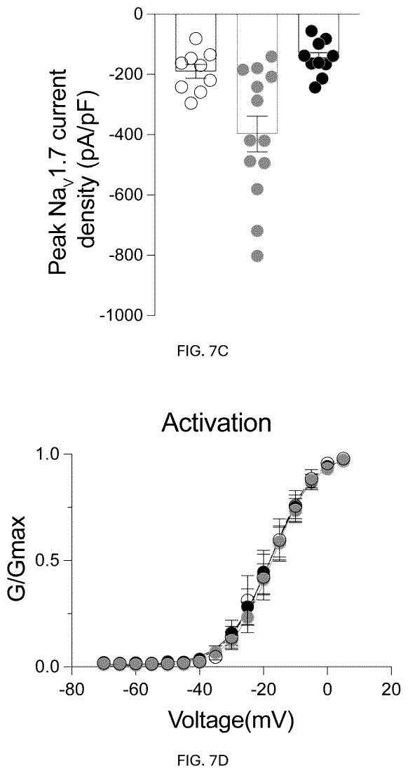

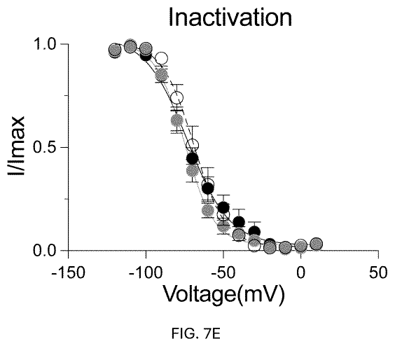

Generally, NaV channels can be divided into nine subtypes that display distinct expression patterns and biological functions in mammals. To clarify whether NRF2 acts on all NaV channels or selectively on a specific subtype, the effects of Compound 1 on NaV1.7 currents in human embryonic kidney (HEK) 293 cells transfected with NaV1.7 plasmid, was observed. Transfection with NaV1.7 ensures that most of the recorded NaV channels are NaV1.7. The results show that treatment of Compound 1 significantly inhibited NaV1.7 current density (FIG. 6). Same inhibiting properties could be observed under excessive oxidative stress condition when HEK293 cells were pre-treated with H₂O₂ (FIG. 7). These results build evidence that Compound 1 is a potent NRF2 activator that relieves neuropathic pain through inhibiting NaV1.7 channels.

### 6.3 Conclusion

Since the NaV1.7 voltage-gated sodium channel has been defined as a central role in the function of nociceptive signal propagation that modulates the generation and progression of neuropathic pain²⁶, targeting NaV1.7 for novel analgesic development is becoming a hotspot in pain research. Here, we Compound 1 was identified as a novel analgesic which shows potential antinociceptive properties in nerve lesion-induced neuropathic pain rodent models. It was further determined that Compound lexerts analgesic abilities through NRF2 activation and NaV1.7 inhibition in DRG neurons. Treatment with Compound 1 can offer a new path for novel analgesic development.

### Exemplary Products, Systems and Methods are Set Out in the Following Items:

1. A method for treating pain in a subject in need thereof, the method comprising administering to the subject an effective amount of a compound having a formula

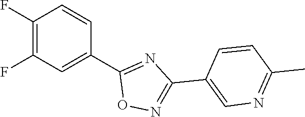 [SMILES: Cc1ccc(-c2noc(-c3ccc(F)c(F)c3)n2)cn1]

or a pharmaceutically-acceptable salt thereof.
2. The method of item 1, wherein the subject has nociceptive pain.
3. The method of any one of the preceding items, wherein the subject has inflammatory pain
4. The method of any one of the preceding items, wherein the subject has neuropathic pain.
5. The method of item 4, wherein the neuropathic pain is peripheral neuropathic pain (PNP).
6. The method of item or 5, wherein the neuropathic pain is a nerve lesion-induced neuropathic pain.
7. The method of any one of the preceding items, wherein the subject has chronic pain.
8. The method of any one of the preceding items, wherein the compound reduces or alleviates allodynia in the subject.
9. The method of any one of the preceding items, wherein the compound reduces or alleviates hyperalgesia in the subject.
10. The method of any one of the preceding items, wherein the pain is associated with a disease of condition.
11. The method of item 10, wherein the disease or condition is selected from the group consisting of cancer, arthritis, osteoarthritis, diabetes, multiple sclerosis, an autoimmune disease, an infection, an injury, a metabolic disease, postoperative pain, post-traumatic pain, post-thoracotomy pain, complex regional pain syndrome (CRPS), thalamic pain, paraplegic pain caused by myelopathy, chemotherapy induced polyneuropathy (CIPN), pain caused by radiation therapy, anesthesia dolorosa, phantom limb pain, small fiber neuropathy (SFN), hereditary motor and sensory neuropathies (HMSN), chronic inflammatory demyelinating polyneuropathy (CIDP), trigeminal neuralgia, post-herpetic neuralgia, intercostal neuralgia, entrapment neuropathies (e.g. carpal tunnel syndrome, tarsal tunnel syndrome, abdominal cutaneous nerve entrapment syndrome), sciatic pain, chronic idiopathic axonal polyneuropathy (CIAP), vulvodynia, proctodynia, Sjogren syndrome, lymphomatous neuropathy, myelomatous neuropathy, carcinomatous neuropathy, and vasculitic/ischaemic neuropathy.
12. The method of item 11, wherein
   the autoimmune disease is selected from the group consisting of lupus, rheumatoid arthritis, Sjogren's syndrome, and Guillain-Barre syndrome;
   the infection is selected from the group consisting of shingles, Lyme disease, hepatitis B and C, leprosy, diphtheria, polio, and HIV;
   the injury is selected from the group consisting of nerve trauma, spinal cord injury and stroke;
   the metabolic disease is selected from the group consisting of kidney disease, liver disease, and hypothyroidism; and
   the entrapment neuropathy is selected from the group consisting of carpal tunnel syndrome, tarsal tunnel syndrome, abdominal cutaneous nerve entrapment syndrome
13. The method of any one of the preceding items, wherein the subject has pain associated with osteoarthritis.
14. The method of any one of the preceding items, wherein the subject has pain associated with an activated voltage-gated sodium channel.
15. The method of any one of the preceding items, wherein the pain is associated with increased oxidative stress in the subject, optionally wherein the compound reduces or alleviates oxidative stress in the subject.
16. The method of any one of the preceding items, wherein the compound inhibits activity of a voltage-gated sodium channel.
17. The method of any one of the preceding items, wherein the compound binds to a voltage-gated sodium channel and has a dissociation constant (Kd) less than about 50 nM.
18. The method of any one of the preceding items, wherein the compound inhibits the activity of a voltage-gated sodium channel and has a half-maximal inhibitory concentration (IC50) of less than about 200 nM.
19. The method of item 18, wherein the voltage-gated sodium channel is NaV1.7.
20. The method of any one of the preceding items, wherein the compound is administered orally, topically, intravenously or intrathecally.
21. The method of any one of the preceding items, wherein the subject is administered a dose of the compound that is effective for achieving a concentration of the compound at the site of action of at least 1 nM.
22. The method of any one of the preceding items, wherein the subject is administered a daily oral dose of the compound of less than about 300 mg.
23. The method of any one of the preceding items, wherein the subject is administered a dosage of about 50 μg/kg/day.
24. The method of any one of the preceding items, wherein the compound activates nuclear factor erythroid 2-related factor 2 (NRF2) in the subject.
25. The method of any one of the preceding items, wherein the effective dose is about 1-100 μg/kg/day per day.
26. A method for treating pain in a subject in need thereof, the method comprising administering to the subject an effective amount of a 1,2,4-oxadiazole derivative, wherein the 1,2,4-oxadiazole compound is represented by the structure:

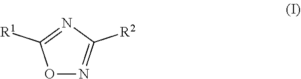

wherein R¹ is an unsubstituted or substituted phenyl or heteroaryl; and R² is an unsubstituted or substituted heteroaryl.
27. A method for treating pain in a subject in need thereof, the method comprising administering to the subject an effective amount of a 1,2,4-oxadiazole derivative that activates nuclear factor erythroid 2-related factor 2 (NRF2), reduces or alleviates oxidative stress in the subject, or inhibits a voltage-gated sodium channel in the subject, wherein the 1,2,4-oxadiazole compound is represented by the structure:

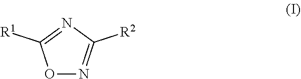

wherein
each of R¹ and R² is independently aryl, heterocyclyl or heteroaryl, each of which may be unsubstituted or substituted;
or a pharmaceutically-acceptable salt thereof.
28. A method for treating pain in a subject in need thereof, the method comprising administering to the subject an effective amount of a 1,2,4-oxadiazole derivative, wherein the 1,2,4-oxadiazole compound is represented by the structure:

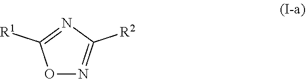

wherein
R¹ is an unsubstituted or substituted phenyl or heteroaryl; and
R² is an unsubstituted or substituted heteroaryl;
or a pharmaceutically-acceptable salt thereof.
29. A method for treating pain in a subject in need thereof, the method comprising administering to the subject an effective amount of a 1,2,4-oxadiazole derivative that activates nuclear factor erythroid 2-related factor 2 (NRF2), reduces or alleviates oxidative stress in the subject, or inhibits a voltage-gated sodium channel in the subject, wherein the 1,2,4-oxadiazole compound is represented by the structure:

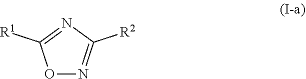

wherein
R¹ is an unsubstituted or substituted phenyl or heteroaryl; and
R² is an unsubstituted or substituted heteroaryl;
or a pharmaceutically-acceptable salt thereof.
30. The method of any one of items 26-29, wherein the compound is selected from the group consisting of

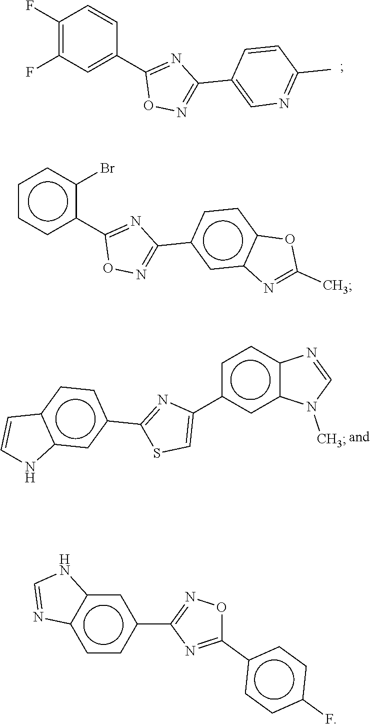

31. The method of any one items 26-30, wherein the compound inhibits the activity of a voltage-gated sodium channel and has a half-maximal inhibitory concentration (IC50) of less than about 200 nM.
32. The method of item 31, wherein the voltage-gated sodium channel is NaV1.7.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

1. Fielding R, Wong W S. Prevalence of chronic pain, insomnia, and fatigue in Hong Kong. Hong Kong Med J 18 Suppl 3, 9-12 (2012).
2. Borsook D, Hargreaves R, Bountra C, Porreca F. Lost but making progress—Where will new analgesic drugs come from ? Sci Transl Med 6, 249sr243 (2014).
3. Yatziv S L, Devor M. Suppression of neuropathic pain by selective silencing of dorsal root ganglion ectopia using nonblocking concentrations of lidocaine. Pain 160, 2105-2114 (2019).
4. Yu F H, Catterall W A. Overview of the voltage-gated sodium channel family. Genome Biol 4, 207 (2003).
5. Nassar M A, et al. Nociceptor-specific gene deletion reveals a major role for Nav1.7 (PN1) in acute and inflammatory pain. Proc Natl Acad Sci USA 101, 12706-12711 (2004).
6. Cox J J, et al. An SCN9A channelopathy causes congenital inability to experience pain. Nature 444, 894-898 (2006).
7. Toledo-Aral J J, et al. Identification of PN1, a predominant voltage-dependent sodium channel expressed principally in peripheral neurons. Proc Natl Acad Sci USA 94, 1527-1532 (1997).
8. Klugbauer N, Lacinova L, Flockerzi V, Hofmann F. Structure and functional expression of a new member of the tetrodotoxin-sensitive voltage-activated sodium channel family from human neuroendocrine cells. EMBO J 14, 1084-1090 (1995).
9. Rush A M, Cummins T R, Waxman S G. Multiple sodium channels and their roles in electrogenesis within dorsal root ganglion neurons. J Physiol 579, 1-14 (2007).
10. Waxman S G, et al. Sodium channel genes in pain-related disorders: phenotype-genotype associations and recommendations for clinical use. Lancet Neurol 13, 1152-1160 (2014).
11. Marchi M, et al. Congenital insensitivity to pain: a novel mutation affecting a U12-type intron causes multiple aberrant splicing of SCN9A. Pain 163, e882-e887 (2022).
12. Zakrzewska J M, et al. Safety and efficacy of a Nav1.7 selective sodium channel blocker in patients with trigeminal neuralgia: a double-blind, placebo-controlled, randomised withdrawal phase 2a trial. Lancet Neurol 16, 291-300 (2017).
13. McDonnell A, et al. Efficacy of the Nav1.7 blocker PF-05089771 in a randomised, placebo-controlled, double-blind clinical study in subjects with painful diabetic peripheral neuropathy. Pain 159, 1465-1476 (2018).
14. Siebenga P, et al. Lack of Detection of the Analgesic Properties of PF-05089771, a Selective Nav 1.7 Inhibitor, Using a Battery of Pain Models in Healthy Subjects. Clin Transl Sci 13, 318-324 (2020).
15. Xu L L, et al. 5-(3,4-Difluorophenyl)-3-(6-methylpyridin-3-yl)-1,2,4-oxadiazole (DDO-7263), a novel Nrf2 activator targeting brain tissue, protects against MPTP-induced subacute Parkinson's disease in mice by inhibiting the NLRP3 inflammasome and protects PC12 cells against oxidative stress. Free Radic Biol Med 134, 288-303 (2019).
16. Decosterd I, Woolf C J. Spared nerve injury: an animal model of persistent peripheral neuropathic pain. Pain 87, 149-158 (2000).
17. Yang H, et al. Reversal of Peripheral Neuropathic Pain by the Small-Molecule Natural Product Narirutin via Block of Na (v) 1.7 Voltage-Gated Sodium Channel. Int J Mol Sci 23, (2022).
18. Mestre C, Pelissier T, Fialip J, Wilcox G, Eschalier A. A method to perform direct transcutaneous intrathecal injection in rats. J Pharmacol Toxicol Methods 32, 197-200 (1994).
19. Chaplan S R, Bach F W, Pogrel J W, Chung J M, Yaksh T L. Quantitative assessment of tactile allodynia in the rat paw. J Neurosci Methods 53, 55-63 (1994).
20. Roth E, Linehan A, Weihrauch D, Stucky C, Hogan Q, Hoben G. Targeted muscle reinnervation prevents and reverses rat pain behaviors after nerve transection. Pain 164, 316-324 (2023).
21. Cai S, et al. Selective targeting of NaV1.7 via inhibition of the CRMP2-Ubc9 interaction reduces pain in rodents. Sci Transl Med 13, eabh1314 (2021).
22. Shan Z, et al. Reversal of Peripheral Neuropathic Pain by the Small-Molecule Natural Product Physalin F via Block of CaV2.3 (R-Type) and CaV2.2 (N-Type) Voltage-Gated Calcium Channels. ACS Chem Neurosci 10, 2939-2955 (2019).
23. Smith B J, McHugh C F, Hirano A A, Brecha N C, Barnes S. Transient and Sustained Ganglion Cell Light Responses Are Differentially Modulated by Intrinsically Produced Reactive Oxygen Species Acting upon Specific Voltage-Gated Na (+) Channel Isoforms. J Neurosci 43, 2291-2304 (2023).
24. Fouda M A, Ghovanloo M R, Ruben P C. Cannabidiol protects against high glucose-induced oxidative stress and cytotoxicity in cardiac voltage-gated sodium channels. Br J Pharmacol 177, 2932-2946 (2020).
25. Kassmann M, et al. Oxidation of multiple methionine residues impairs rapid sodium channel inactivation. Pflugers Arch 456, 1085-1095 (2008).
26. Meents J E, et al. The role of Nav1.7 in human nociceptors: insights from human induced pluripotent stem cell-derived sensory neurons of erythromelalgia patients. Pain 160, 1327-1341 (2019).
27. Target Fishing Reveals a Novel Mechanism of 1,2,4-Oxadiazole Derivatives Targeting Rpn6, a Subunit of 26S Proteasome. https://pubs.acs.org/doi/10.1021/acs.jmedchem.1c02210
28. 5-(3,4-Difluorophenyl)-3-(6-methylpyridin-3-yl)-1,2,4-oxadiazole (DDO-7263), a novel Nrf2 activator targeting brain tissue, protects against MPTP-induced subacute Parkinson's disease in mice by inhibiting the NLRP3 inflammasome and protects PC12 cells against oxidative stress. https://www.sciencedirect.com/science/article/abs/pii/S0891584918323232?via % 3Dihub

What is claimed:

1. A method for treating pain in a subject in need thereof, the method comprising administering to the subject an effective amount of a compound having a formula 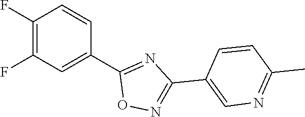 [SMILES: Cc1ccc(-c2noc(-c3ccc(F)c(F)c3)n2)cn1]
or a pharmaceutically-acceptable salt thereof.

2. The method of claim 1, wherein the pain is nociceptive pain.

3. The method of claim 1, wherein the pain is inflammatory pain.

4. The method of claim 1, wherein the pain is neuropathic pain.

5. The method of claim 4, wherein the neuropathic pain is peripheral neuropathic pain (PNP).

6. The method of claim 4, wherein the neuropathic pain is a nerve lesion-induced neuropathic pain.

7. The method of claim 1, wherein the pain is chronic pain.

8. The method of claim 1, wherein the compound reduces or alleviates allodynia in the subject.

9. The method of claim 1, wherein the compound reduces or alleviates hyperalgesia in the subject.

10. The method of claim 1, wherein the pain is associated with a disease of condition.

11. The method of claim 10, wherein the disease or condition is selected from the group consisting of cancer, arthritis, osteoarthritis, diabetes, multiple sclerosis, an autoimmune disease, an infection, an injury, a metabolic disease, postoperative pain, post-traumatic pain, post-thoracotomy pain, complex regional pain syndrome (CRPS), thalamic pain, paraplegic pain caused by myelopathy, chemotherapy induced polyneuropathy (CIPN), pain caused by radiation therapy, anesthesia dolorosa, phantom limb pain, small fiber neuropathy (SFN), hereditary motor and sensory neuropathies (HMSN), chronic inflammatory demyelinating polyneuropathy (CIDP), trigeminal neuralgia, post-herpetic neuralgia, intercostal neuralgia, entrapment neuropathies (e.g. carpal tunnel syndrome, tarsal tunnel syndrome, abdominal cutaneous nerve entrapment syndrome), sciatic pain, chronic idiopathic axonal polyneuropathy (CIAP), vulvodynia, proctodynia, Sjogren syndrome, lymphomatous neuropathy, myelomatous neuropathy, carcinomatous neuropathy, and vasculitic/ischaemic neuropathy.

12. The method of claim 11, wherein
the autoimmune disease is selected from the group consisting of lupus, rheumatoid arthritis, Sjogren's syndrome, and Guillain-Barre syndrome;
the infection is selected from the group consisting of shingles, Lyme disease, hepatitis B and C, leprosy, diphtheria, polio, and HIV;
the injury is selected from the group consisting of nerve trauma, spinal cord injury and stroke;
the metabolic disease is selected from the group consisting of kidney disease, liver disease, and hypothyroidism; and
the entrapment neuropathy is selected from the group consisting of carpal tunnel syndrome, tarsal tunnel syndrome, abdominal cutaneous nerve entrapment syndrome

13. The method of claim 1, wherein the compound activates nuclear factor erythroid 2-related factor 2 (NRF2) in the subject.

14. The method of claim 1, wherein the pain is associated with increased oxidative stress in the subject.

15. The method of claim 14, wherein the compound reduces or alleviates oxidative stress in the subject.

16. The method of claim 1, wherein the compound inhibits activity of a voltage-gated sodium channel.

17. The method of claim 16, wherein the compound binds to the voltage-gated sodium channel with a dissociation constant (K_{d}) of less than about 50 nM.

18. The method of claim 16, wherein the compound has a half-maximal inhibitory concentration (IC₅₀) of the voltage-gated sodium channel of less than about 200 nM.

19. The method of claim 16, wherein the voltage-gated sodium channel is NaV1.7.

20. The method of claim 1, wherein the compound is administered orally, topically, intravenously or intrathecally.

21. The method of claim 1, wherein the subject is administered a dose of the compound that is effective to achieve a concentration of the compound at the site of action of at least about 1 nM.

22. The method of claim 1, wherein the effective dose is a daily oral dose of less than about 300 mg.

23. The method of claim 1, wherein the effective dose is about 1-100 μg/kg/day per day.

24. A method for treating pain in a subject in need thereof, the method comprising administering to the subject an effective amount of a 1,2,4-oxadiazole derivative that activates nuclear factor erythroid 2-related factor 2 (NRF2), reduces or alleviates oxidative stress in the subject, or inhibits a voltage-gated sodium channel in the subject, wherein the 1,2,4-oxadiazole compound is represented by the structure: 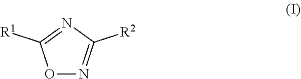
wherein
each of R¹ and R² is independently aryl, heterocyclyl or heteroaryl, each of which may be unsubstituted or substituted;
or a pharmaceutically-acceptable salt thereof.

25. The method of claim 24, wherein the compound is selected from the group consisting of

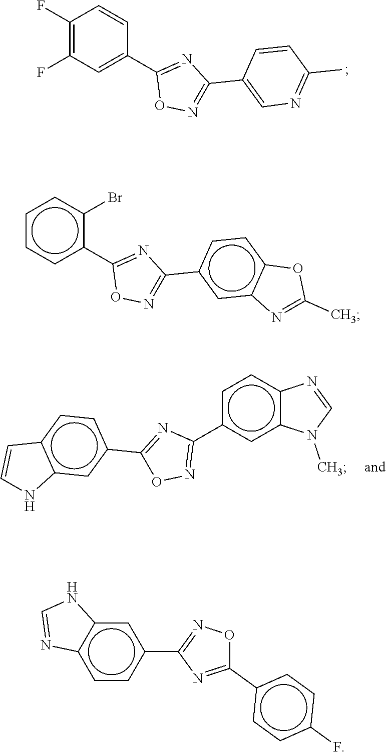

26. The method of claim 24, wherein the compound inhibits the activity of the voltage-gated sodium channel with half-maximal inhibitory concentration (IC50) of less than about 200 nM.

27. The method of item 24, wherein the voltage-gated sodium channel is NaV1.7.
